(19) United States
(12) Patent Application Publication
(10) Pub. No.: US 2003/0176660 A1
(43) Pub. Date: Sep. 18, 2003

(54) Immunoglobulin purification

(76) Inventors: Thomas C. Ransohoff, Lexington, MA (US); Owen J. Murphy, Worcester, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/360,888

(22) Filed: Feb. 7, 2003

Related U.S. Application Data
(60) Provisional application No. 60/355,182, filed on Feb. 8, 2002.

(51) Int. Cl. C07K 16/44; C07K 16/00

(52) U.S. Cl. 530/388.15; 800/6

(57) ABSTRACT

The invention features compositions containing avian-derived transgenic non-avian antibodies and methods of recovering the compositions from transgenic avian eggs. Chromatographic techniques permit the purification of non-avain proteins produced in an avain tissue from contaminating avian tissue components for administration to humans.

### RELATED APPLICATIONS

This application claims priority to U.S. S. No. 60/355,182, filed Feb. 8, 2002, the entire contents of which is hereby incorporated by reference.

### FIELD OF THE INVENTION

The invention relates to methods of isolating immunoglobulins.

### BACKGROUND OF THE INVENTION

Avian transgenic animals can be used to produce proteins in the white (albumen) fraction of transgenic chicken eggs. However, the proteins must be purified from avian components for use, e.g., administration to humans, for the treatment of disease. Adequate purification schemes have not been developed.

### SUMMARY OF THE INVENTION

The invention provides purification methods, which yield a substantially pure recombinant polypeptide suitable for human administration, and features compositions containing a non-avian protein, e.g., a human or humanized antibody molecule, which has been produced by an avian tissue but is substantially purified from avian proteins. For example, for therapeutic use, a non-avian protein or polypeptide produced in an avian tissue must be substantially free of avian proteins, which may cause an allergic reaction in a human patient. A protein such as an antibody is substantially free of naturally-occurring avian associated components when it is separated from those impurities which accompany it in an avian egg. The non-avian transgenic antibody preparation is purified from host avian (e.g., chicken) immunoglobulins. By substantially pure is meant that the composition contains at least 85, 90, 95, 97, 99, or 100 percent w/w of a non-avian protein or polypeptide compared to avian proteins.

Accordingly, the invention includes a composition containing an avian-derived transgenic non-avian antibody in which less than 1000 ppm of the composition is an avian antibody. Preferably, the composition contains less than 100 ppm, more preferably less than 10 ppm, or less than 1 ppm, of an avian antibody. The avian animal is a chicken and the non-avian antibody is a human antibody. For example, a transgenic chicken containing a nucleic acid encoding human antibody molecules under the control of an albumen-specific promoter (e.g. an ovalbumen promoter) produces eggs, which contain the human gene product. A transgenic chicken egg contains at least 10 mg of human antibody per egg. For example, the egg contains 50 mg of human antibody (approximately 2 mg/ml of human antibody).

The non-avian antibody contains an amino acid sequence of a human immunoglobulin Fc portion. For example, the non-avian antibody is a humanized antibody. The composition (e.g., egg albumen) contains at least 0.1 g/L, preferably at least 1 g/l of the non-avian antibody, more preferably at least 3-10 g/l of the non-avian antibody, e.g., the concentration of non-avian antibody is at least 1 g/l of egg albumen. A transgenic hen's egg contains approximately 0.1 mg/ml of chicken antibody prior to purifying the non-avian antibody.

The antibody is a transgenic monoclonal antibody or a fragment thereof. The purified protein is intact monoclonal antibody, or an Fe fusion protein, or an immunologically-active antibody fragment containing an Fe portion. Chimeric antibody molecules, e.g., an antibody which contains the binding specificity of one antibody, e.g., of murine origin, and the remaining portions of another antibody, e.g., of human origin, are also purified using the methods described herein.

Also within the invention is a method for recovering a recombinant non-avian antibody from a transgenic avian egg by contacting the albumen fraction of the transgenic egg with a ligand, which preferentially binds to an Fe portion of the non-avian antibody compared to an avian antibody. For example, the dissociation constant for binding of the ligand to a human antibody is at least 10-fold lower than the dissociation constant for ligand binding to a chicken antibody. More preferably, the dissociation constant for ligand binding to a human antibody is at least 100-fold or 1000-fold lower than for ligand binding to a chicken antibody.

The ligand binds to the cleft between a CH2 and CH3 domain of a non-avian antibody. For example, the ligand is staphylococcal protein A, protein G, or a protein or organic molecule, which binds to a human antibody at the same site as protein A or G. The method allows purification of a transgenic human antibody produced in a hen's egg from contaminating chicken antibodies and other chicken contaminants in egg albumen.

The egg albumen fraction of an egg is purified from the yolk fraction. Optionally, the albumen fraction is homogenized or processed using a mild precipitation technique to remove certain egg proteins without causing the antibody to precipitate. For example, homogenization is carried out at low shear rate to preserve the integrity of the antibody sought to be purified. The albumen fraction containing transgenic human IgG as well as chicken (or other avian) immunoglobulins is contacted with the ligand, and the transgenic IgG purified using affinity chromatographic techniques. The wash and elution buffers allow elution of the human antibody to yield a composition, which contains less than 1000 ppm avian antibody relative to human IgG. The initial preparation comprises a composition containing approximately 100,000 ppm avian antibody relative to human IgG. Therefore, the method provides at least 2 log clearance of contaminating avian antibodies from human IgG; preferably at least 4 log clearance is obtained.

The invention also includes a method of detecting an avian immunoglobulin, e.g., a chicken IgG, in a mixture of heterologous non-avian antibodies. For example, the method allows detection of minute quantities of chicken IgG in egg albumen, e.g., 1-10 μg/ml by Western blot and less than about 10 ng/ml by ELISA.

Other features and advantages of the invention will be apparent from the following description of the preferred embodiments thereof.

### BRIEF DESCRIPTION OF THE DRAWINGS

FIG. 1. is a photograph of an SDS-PAGE electrophoretic gel and Western Blot Assay showing detection of endogenous chicken IgG in yolk and egg white.

Lanes 1 and 2 show proteins from egg yolk and egg white (respectively) on a Coomasie Blue stained gel. Lanes 3-7 show the result of a Western Blot Assay to detect endogenous chicken IgG using a rabbit anti-chicken IgG antibody linked to alkaline phosphatase.

### DETAILED DESCRIPTION

The methods described herein permit isolation of certain non-avian proteins produced in an avian tissue (e.g., a hen's egg) from contaminating avian tissue components. For example, a human antibody was purified from egg albumen. Human immunoglobulins such as those of the IgG isotype were isolated from a solution containing antibodies from a heterologous species such as a chicken using chromatographic techniques. Human IgG is purified from chicken IgG using a protein A column. The binding and elution conditions are such that the dissociation constant for binding of the ligand to a human antibody is at least 10-fold lower than the dissociation constant for ligand binding to a chicken antibody. The dissociation constant for S. Aureus Protein A is known in the art, e.g, as reported by Li et al (Nature Biotechnol. 16, 190-195 (1998)) is 7×10⁻⁸ M.

Harsh precipitation and extraction techniques such as those using strong acids, or high concentrations of solvents are not used because they may destroy the structure and antigen specific binding capability of the antibody. Affinity separations, are used as a means for providing the high selectivity needed for removing the majority of egg proteins from an antibody product.

To evaluate and monitor yield of the purification method, human IgG derived from serum was spiked into test solutions such as egg albumen. SDS-PAGE results indicate that Protein A (Repligen IPA-400-HC),which binds to the CH2/CH3 region of human IgG effectively recover human antibody from egg albumen with good selectivity.

A wash/binding Buffer contains 25 mM Tris, 125 mM NaCl, 5 mM EDTA, pH 7.2. Elution Buffer contains 0.1 M acetic acid. Alternatively, the wash/binding buffer contains phosphate buffered saline (PBS) and glycine.

Development of wash steps and optimization of binding conditions affects selectivity and fold-clearance. For example, the following elution conditions are optionally used: 1) high salt (e.g., 1M NaCl), and alternative salts (e.g., CaCl₂); 2) high non-ionic surfactant (e.g., 0.1% Triton X-100); 3) modest concentrations of denaturants and chaotropes (e.g., 1M Urea or 0.5M GuHCl); 4) mild acid or base washes (e.g., pH 5-9), optionally with small amounts of solvent (e.g., 5-10% EtOH); and 5) hydrophobic modifiers, such as ethylene glycol.

Other ligands, which bind to an epitope in the same or similar region of IgG, are also useful. Protein A binds to the Fc portion of IgG, while Protein G binds preferentially to the Fc portion of IgG but can also bind to the Fab region, making it useful for purification of F(ab)′2 fragments of IgG. Additionally, the use of expanded bed adsorption (EBA) is an alternative to column chromatography to minimize fouling with the very high protein load.

Methods of Quantitating Immunoglobulin Purification

To monitor the level of purification using the antibody isolation methods, a quantitative assay was developed for human IgG. Reverse phase and cation exchange both proved unsatisfactory for the polyclonal human IgG initially tested, therefore, an SEC-based assay was used. An assay was developed using Tosoh Biosep's TSK-GEL 3000 SWXL column with detection at 280 nm. Sensitivity was better than 10 μg/injection in relatively pure samples (>80% purity by HPLC).

Using this assay, yields were determined for purification of human IgG from egg albumen an affinity media, which binds to the CH2/CH3 domain of human IgG (initial concentration 1 mg/ml hIgG in EWP; loadings=2.5-4 mg IgG/ml column volume). From 2 runs using Protein A media IPA-400 HC, essentially quantitative recovery of IgG was achieved in the eluted peak by adding up amounts recovered in all eluted fractions.

Western blot assays were used to demonstrate that a small quantity of chicken IgG is detectable in egg albumen. This assay allows reliable, sensitive and accurate monitoring of chicken Ig in human IgG preparations. While the presence of chicken IgG is a challenge for purification development for monoclonals from egg albumen, the initial levels of chicken IgG are low by comparison to levels in processes where host-human IgG separations have already been achieved (serum-containing cell culture processes and milk-based processes).

### EXAMPLE 1

### Protein A-Linked Resins Offer the Effective Separation of Human IgG Antibodies from Endogenous Chicken IgG Present in Egg White

Western Blotting of blotted antigen was carried out. Detection was accomplished using standard alkaline phosphatase-based cleavage of a chemiluminescent substrate. A Western Blot screen was carried out for chicken IgG in egg yolk (as a positive control), thin-white and thick-white fractions using an alkaline phosphatase conjugated rabbit anti-chicken IgG antibody.

12.5 nL of an egg fraction (yolk, thin-white or thick-white) is defined as the diluted amount loaded on an SDS-polyacrylamide gel electrophoresis (SDS-PAGE) based on a starting amount of whole fraction.

After SDS gel electrophoresis, proteins were electrotransferred to a nitrocellulose membrane for 2 hours at 75 V constant. Membranes were blocked overnight with constant agitation in a blocking buffer consisting of 0.2% casein/0.2% Tween 20/Phosphate buffered saline (PBS). The membranes were then incubated with a 1/20000 dilution of rabbit anti-chicken IgG antibody linked to alkaline phosphatase in block buffer for 1-2 hrs. at room temperature. The membranes were then washed and processed according to the Tropix Western-Light procedure (Tropix, Inc. is a subsidiary of Applied Biosystems located at 47 Wiggins Ave., Bedford, Mass.).

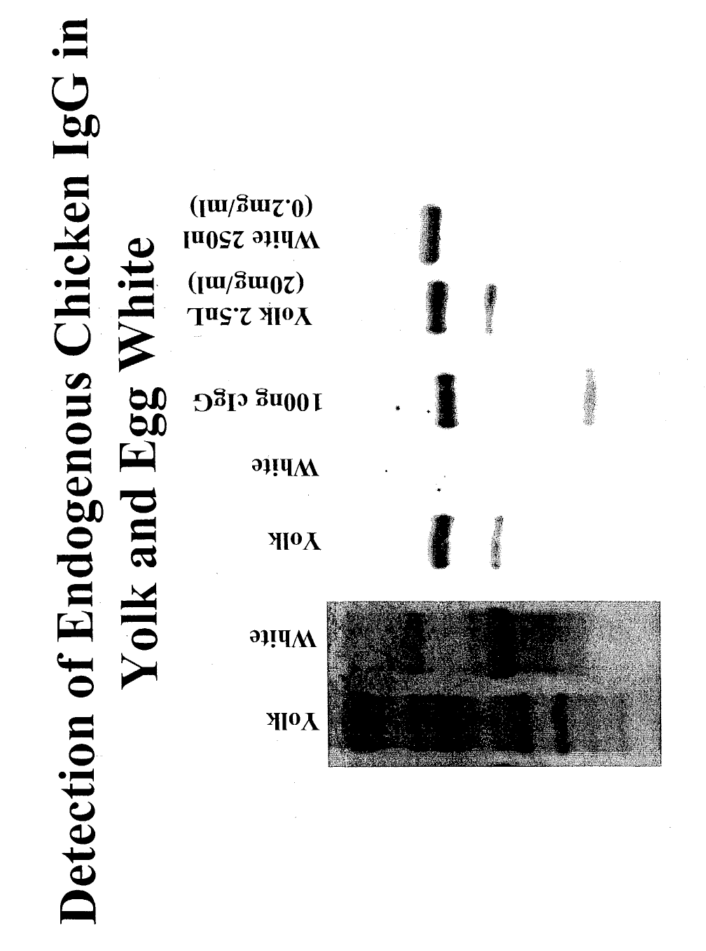

A strong signal band was seen for the IgG heavy and light chains in the egg yolk fraction as expected. A much weaker signal was seen for the IgG heavy and light chain bands in the egg-white fractions indicating that chicken IgG is present in the egg-white fractions and its concentration is roughly the same in the thick and thin portions of the egg white. But a much lower concentration of chicken IgG is present in the egg white than in the yolk. A typical Western is shown in FIG. 1.

Immunoprecipitation of Chicken IgG from Egg Yolk and Thin-White Fractions

IgG was immunoprecipitated from the egg albumen fraction of a hen's egg. Egg albumen contains a thick layer and a thin layer. Thick white was not run initially because an equal distribution of chicken IgG has been detected in the thin and thick fractions.

Immunoprecipitation was carried out using the less viscous thin fraction to assess the relative volumes needed of each for approximately equal IgG concentrations. The IP was performed using Protein A-Agarose beads followed by Western blot immunodetection using alkaline-phosphatase conjugated rabbit anti-chicken IgG antibody. A titration curve of yolk and egg white fractions was immunoprecipatated with protein A and immunodetected.

A protocol was used with Tween 20 washes and Tween 20 included in the initial binding interaction between protein A and chicken IgG. Aliquots of sample (yolk or egg-white) were diluted in 1 mL of PBS/0.3% Tween 20. Then aliquots were taken from each of the starting 1 mL stocks and 20 μL of Protein A-agarose beads was added. The mixture was incubated gentle agitation and pelleted in a microcentrifuge. The pellet was washed 3 times with 200 μL of PBS/0.3% Tween 20, pelleted again and resuspended in an equal volume of SDS-PAGE sample buffer.

Immunodetection was run as described above. The heavy chain band intensity of 5 nL, 10 nL or 20 nL of yolk fraction was approximately equivalent to 500 nL, 1 μL or 2 μL of the respective thin-white fractions. The egg white chicken IgG concentration is approximately 100-fold less concentrated than in the yolk fraction. Due to the IP with protein A, the band intensity of 10 nL of yolk was quite reduced compared to the non-IP 12.5 nL in the original experiment. The same held for the comparative intensities in thin-white heavy chain bands between the non-IP experiment and the IP experiments.

The data indicated a negative effect on chicken IgG clearance using protein A beads. Although it is known that protein A has a high affinity for human antibodies and a lesser affinity for other antibodies, the high degree of separation of human monoclonals produced in the egg white from the endogenous chicken population based on protein A interactions was surprising.

To affirm the effect of protein A based IP on chicken IgG clearance, the next determination was performed using IP and non-IP conditions performed on the same set of samples. Immunoprecipitation of chicken IgG from egg yolk, thin white, and a known concentration of standard chicken IgG was performed in parallel with a straight titration of yolk, thin-white and standard chicken IgG to assess the negative effect of protein A precipitation on chicken IgG concentration. The IP was performed using Protein A-Agarose beads followed by Western blot immunodetection using alkaline-phosphatase conjugated rabbit anti-chicken IgG antibody. A titration curve of yolk and egg white fractions was immunoprecipatated with protein A or not and immunodetected.

The non-IP fractions were prepared as follows. A chicken IgG standard titration curve consisting of 250 ng and 500 ng gel loads was prepared by dilution with PBS and SDS sample buffer. Titrations of 2.5 nL, 5 nL and 10 nL yolk fractions were prepared by dilution with PBS and SDS sample buffer and gel loaded. Titrations of 500 nL, 1.0 μL and 2.5 μL thin-white fractions were prepared by dilution with PBS and SDS sample buffer and gel loaded. The IP fractions were prepared as described above. Tween 20 was included in the initial binding interaction between protein A and chicken IgG. Aliquots of sample (yolk or egg-white) were diluted in 1 mL of PBS/0.3% Tween 20. Then aliquots were taken from each of the starting 1 mL stocks and 20 μL of Protein A-agarose beads was added. The mixture was incubated gentle agitation and pelleted in a microcentrifuge. The pellet was washed 3 times with 200 μL of PBS/0.3% Tween 20, pelleted again and resuspended in an equal volume of SDS-PAGE sample buffer. Immunodetection proceed as described above. An IP chicken IgG standard titration curve having an expected 250 ng and 500 ng of gel loads was prepared. Egg yolk was immunoprecipitated and gel loads expected to give 2.5 nL, 5.0 nL and 10.0 nL of yolk were prepared. Egg thin white was immunoprecipitated and gel loads expected to give 500 nL, 1.0 μL and 2.5 μL of thin white were prepared.

The non-IP conditions produced bands of high intensity and the IP conditions drastically reduced band intensity. These data confirmed the results indicating that the precipitation step with protein A does not bring down the chicken IgG fraction with high affinity and most of the fraction is left behind in the supernatant.

### EXAMPLE 2

### Methods for Purification of Heterologous Mammalian IgG Products from Egg Albumen Clearance Calculations for Chicken Ig from Human IgG

Target expression levels for monoclonal antibodies in egg albumen of transgenic chickens are greater than 50-100 mg/egg. At 50 mg/egg, and assuming egg white volume of 25 ml, the heterologous IgG concentration would be 2 mg/ml in the egg albumen.

The chicken Ig concentration in egg albumen fraction was determined from western blot assays described above to be approximately 100-fold lower than the concentration in yolk. The concentration of IgY in chicken egg yolk is estimated at approximately 10 g/L (cf. Egg Science and Technology, 4^{th} ed., Stadelman W J, and Cotterill O J, eds., Food Products Press, NY, 1995, p. 149); therefore, the initial concentration of chicken Ig in egg albumen is approximately 0.1 mg/ml. Thus, the initial relative concentration of chicken to human Ig would be approximately 50,000 ppm.

$$\\ \begin{matrix}Rel . concentration ( ppm ) = 1 E6 * ( ( 0.1 g \text{/} L ) / ( 2 g \text{/} L ) \\ = 50 , 000 ppm\end{matrix}$$

The level of chicken Ig that is acceptable in a human antibody therapeutic product depends on a number of factors, including therapeutic dose and dosing regimen, clinical indication, and the immunogenicity of chicken Ig. Acceptable levels of potentially imunogenic proteins are in the low ppm level range. Compositions suitable for administration to human contain chicken Ig levels below 5 ppm. The methods described herein provide fro a log reduction factor for chicken Ig (relative to the human IgG product) of greater than 4 logs.

$$\begin{matrix}LRF = log ( Initial rel . concentration \text{/} Final rel . concentration ) \\ = log ( 50 , 000 ppm \text{/} 5 ppm ) \\ = 4\end{matrix}$$

To achieve significant safety margins, the process preferably yields a LRF value of greater than 4. LRF for processes are generally determined by multiplying LRF values for different process steps, assuming these steps have orthogonal separation mechanisms. For many processes, it is possible to envision multiple separation mechanisms, each with reasonably high levels of clearance. However, in the case of separating chicken Ig from human Ig, it is unlikely that high levels of clearance will be achieved with separation methods relying on charge and size. Therefore, the methods described herein are based on an affinity process step that can deliver LRF values approaching the requirements of a composition suitable for human administration.

### EXAMPLE 3

### Homogenization of Egg Albumen

The egg albumen fraction of a chicken egg was separated from egg yolk using an standard egg separator. This is appropriate to large-scale processing of egg albumen because large-scale separators use similar cups for separation of albumen from yolk are commercially available. The recovered albumen fraction was heterogeneous, containing some solids, a “thick” fraction, and a “thin” fraction. The thick fraction is relatively viscous, having a viscosity of approximately 20 centapois (cp) at a shear rate of 24 inverseconds (s⁻¹). By reducing the viscosity of the thick fraction and making the solution homogeneous, the ability to manage the egg albumen during further processing was improved.

The data described herein demonstrates the ability to reduce viscosity and provide a homogeneous solution using mechanical mixing at surprisingly low shear rates. Initial work on homogenization was conducted using a microfluidizer (Microfluidics, Inc., Newton, Mass.). Egg albumen was processed on a Microfluidizer M-110Y using 1-3 passes in processing conditions ranging from 2 kpsi to 20 kpsi. All conditions provided a homogeneous effluent with a viscosity of approximately 1 cp. Material processed at 10 kpsi was filtered more readily through a Cuno Zeta Plus 05SP depth filter than material processed at 2 kpsi; however, both conditions provided a homogeneous solution with a viscosity close to water that would be suitable for further processing. Shear rates for microfluidizers are relatively high, ranging from 5.1 to 6.8×10⁶ s⁻¹. Although this method is effective at homogenization, some proteins/polypeptides may be rendered denatured or otherwise compromised (e.g., lower specific activity) with such high shear rates

Low shear rate homogenization is desirable to minimize damage to the protein product. Surprisingly, a homogeneous solution with viscosity close to water was achieved for approximately 30 ml of egg albumen with a lab stir-plate mixer operating for at a shear rate of approximately 23 s⁻ for about 60 minutes.

The data indicated that viscosity reduction and homogenization is achievable using a relatively gentle mechanical mixing technique, e.g., one with a low shear rate, such as static mixing.

### EXAMPLE 4

### Clarification of Egg Albumen

Prior to filtration, egg albumen is processed to (1) reduce viscosity, and (2) remove insoluble stringy matter. Removal of insoluble stringy matter improves filtration (e.g., reduces clogging of the filter) and improves chromatography (e.g., reduces column clogging).

Ovomucin is a glycoprotein in egg albumen, contributes to the gel-like consistency and viscosity of albumen. This glycoprotein may also form strings in the albumen fraction. While homogenization reduces viscosity and provides a homogeneous liquid, the resulting egg albumen solution, even after dilution with up to 10 volumes of a suitable buffer, such as PBS, is capable of plugging membrane filters (dead-end and tangential flow), chromatography columns, and even expanded bed adsorption devices. Ovomucin in the albumen fraction plays a role in column clogging.

A mild acid, low conductivity precipitation technique further improves filterability. For example, precipitation using an acid such as acetic acid (pH 4-5) successfully reduces the amount ovomucin in the egg albumen and improves filterability, which in turn, improves purification of transgenic non-avian antibodies using the affinity chromatography techniques described below or alternatively using anion or cation exchange chromatography as an initial purification step (e.g., an anion exchange method is described in Selected Methods in Cellular Immunology, Barbara B. Mishell and Stanley M. Shiigi, eds., Chap. 12 “Purification of Immunoglobulins and Their Fragments,” pp. 278-281, W H Freeman & Co., San Francisco, Calif.).

### EXAMPLE 5

### Clearance of Chicken Ig from Human Ig

Experiments were carried out to test the ability of immobilized Protein A (IPA-400 HC, Repligen Corp., Needham, Mass.) to clear chicken Ig from human Ig. The level of human Ig in purified fractions was determined using SEC HPLC (TSK-GEL 3000 SWXL); the level of chicken Ig was estimated using a Western blotting procedure described above.

The sample for the experiments was prepared by spiking serum-derived human IgG(Sigma P/N 14506) into egg albumen from freshly cracked eggs. The initial level of human IgG in the egg white was 1 mg/ml. The load for the Protein A column (PROTEIN A LOAD) was prepared by diluting the spiked egg albumen 10-fold with Protein A equilibration buffer (25 mM Tris, 125 mM NaCl, 5 mM EDTA, pH 7.2) and filtering the resulting solution.

The Protein A column was eluted with 0.1N HOAc after washing with a) only equilibration buffer or b) equilibration buffer and PBST (phosphate-buffered saline with 0.05% Tween-20). The eluted peak was collected in fractions for analysis (PROTEIN A PEAK). The concentration of human IgG (hIgG) in the main eluted peak fraction and estimated overall hIgG yield (estimated by adding amounts from all eluted fractions) for each run is provided below:

TABLE 1

| Wash Condition | Conc hIgG Pk. Fraction (μg/m) | Est'd Yield |
|---|---|---|
| Equil. Buffer only | 1100 | 80% |
| Equil. Buffer + PBST | 1390 | 100% |

Chicken IgG Protein A Binding

Experiments were carried out to evaluate chicken IgG binding to protein A. Load sample, fractions collected from the protein A column, wash samples (first and second wash), and flow through samples were tested.

Samples were diluted with one volume of SDS sample buffer and loaded 5 μL per gel lane. The proteins on the gel was immunoblotted according to the same Western procedure described. Along with the column samples, 100 ng of Human IgG was included as a negative control and 100 ng of chicken IgG was included as a positive control for the rabbit anti-chicken IgG-alkaline phosphatase Ab.

Western blots were run on the PROTEIN A LOAD, PROTEIN A FT (material that did not bind to the Protein A column during loading) and PROTEIN A PEAKS from the two runs. Chicken Ig standards of 100 ng, 10 ng and 1 ng per lane were run on the Western blot. Both heavy and light chains are clearly visible in the 100 ng lane; heavy chain only is near the limit of detection in the 10 ng lane; and no heavy or light chain can be detected in the 1 ng lane. Therefore, the limit of detection for this assay is estimated to be approximately 10 ng/lane (heavy chain only). The estimated amounts of chicken Ig in the various lanes are as follows.

TABLE 2

| | Vol. | Est'd | Est'd |
|---|---|---|---|
| Sample | Loaded (μL) | Amt. (ng) | Conc. (μg/ml) |
| PROTEIN A LOAD | 2.5 | 50 | 20 |
| PROTEIN A FT (n = 2) | 2.5 | 50 | 20 |
| PROTEIN A PEAK1 | 10 | ≦10 | ≦1 |
| (Equil buffer only) | | | |
| PROTEIN A PEAK2 | 10 | ≦10 | ≦1 |
| (Equil buff + PBST) | | | |

“≦” indicates less than or equal to.

The only noticeable bands detected with the anti-chicken IgG antibody were in the original load sample lane and the first wash sample lane. No noticeable chicken IgG bands were seen in any of the other lanes corresponding to further wash and elution steps from the protein A column. Furthermore, the light and heavy chains of the 100 ng chicken IgG lane was intense and no cross reactivity was seen with the human IgG (negative control). These results were surprising. Separation of human monoclonals antibodies produced in chicken eggs was achieved using Protein A with little or no cross-reactivity.

The chicken Ig concentration of 20 μg/ml in the PROTEIN A LOAD is within a factor of 2 from the calculations described above (100 μg/ml with a 10-fold dilution would be 10 μg/ml). Based on the data from above, the relative concentrations of chicken Ig to hIgG in the LOAD and PEAK samples and a log reduction factor (LRF) for chicken Ig is determined.

TABLE 3

| | | Chick Ig | hIgG | | |
|---|---|---|---|---|---|
| | | Conc. | Conc. | hIgG | Rel. |
| Sample | Vol. (ml) | (μg/ml) | (μg/ml) | Amt. (mg) | conc. (ppm) |
| RUN 1: | | | | | |
| LOAD | 50 | 20 | 100 | 5.0 | 200,000 |
| PEAK1 FXN | 2.5 | ≦1 | 1100 | 2.8 | ≦910 |
| RUN 2: | | | | | |
| LOAD | 50 | 20 | 100 | 5.0 | 200,000 |
| PEAK2 FXN | 2.5 | ≦1 | 1390 | 3.5 | ≦720 |

“≦” indicates less than or equal to.

The data indicated that the methods described herein achieve a LRF of at least 2 for chicken Ig relative to human IgG using immobilized Protein A.

TABLE 4

| | |
|---|---|
| Washing with Equil buffer only: LRF ≧ | log (200,000/910) = 2.3 |
| Washing with Equil buffer + PBST: LRF ≧ | log (200,000/720) = 2.4 |

“≧” indicates less than or equal to.

Other embodiments are within the following claims.

What is claimed is:

1. A composition comprising an avian-derived transgenic non-avian antibody, wherein said composition comprises less than 10 ppm of an avian antibody.

2. The composition of claim 1, wherein said composition comprises less than 1 ppm of an avian antibody.

3. The composition of claim 1, wherein said avian is a chicken.

4. The composition of claim 1, wherein said non-avian antibody is a human antibody.

5. The composition of claim 1, wherein said non-avian antibody comprises an amino acid sequence of a human immunoglobulin Fc portion.

6. The composition of claim 1, wherein said non-avian antibody is a humanized antibody.

7. The composition of claim 1, wherein said non-avian antibody comprises at least 0.1 g/l of said composition.

8. The composition of claim 1, wherein said non-avian antibody comprises at least 3-10 g/l of said composition.

9. A method for recovering a recombinant non-avian antibody from a transgenic avian egg, comprising contacting the albumen fraction of said egg with a ligand which preferentially binds to an Fc portion of said non-avian antibody compared to an avian antibody.

10. The method of claim 9, wherein said ligand binds to the cleft between a CH2 and CH3 domain of said non-avian antibody.

11. The method of claim 9, wherein said ligand is protein A.

12. The method of claim 9, wherein said ligand is protein G.

13. The method of claim 9, wherein said non-avian antibody is a human antibody.

14. The method of claim 9, wherein said non-avian antibody is a humanized antibody.

15. The method of claim 9, further comprising homogenizing said albumen fraction.
